(19) United States
(12) Patent Application Publication
(10) Pub. No.: US 2017/0220766 A1
(43) Pub. Date: Aug. 3, 2017

(54) SYSTEM AND METHOD FOR DOSAGE STANDARDIZATION

(71) Applicant: Michelle Mangione, Dana Point, CA (US)

(72) Inventor: Michelle Mangione, Dana Point, CA (US)

(21) Appl. No.: 15/159,731

(22) Filed: May 19, 2016

Related U.S. Application Data
(60) Provisional application No. 62/290,103, filed on Feb. 2, 2016.

(51) Int. Cl. G06F 19/00; A61K 36/185; G01N 33/00; A61K 31/352

(52) U.S. Cl. CPC G06F 19/3456; A61K 31/352; A61K 36/185; G01N 33/0098

(57) ABSTRACT

Disclosed is a method and system for standardizing ingredients whereby a user creates a profile comprising at least the user tolerance of an active ingredient. A system prompts user input by providing one or more options for a recipe creation. The user selects at least one of the options and a desired effect of the recipe. The system determines the composition of the recipe and an algorithm creates a threshold using the user characteristics and the desired effect. The algorithm adjusts the amount of recipe components based on a deviation of the user characteristics from threshold values. The system generates instruction for recipe preparation. The system also comprises an interactive apparatus using multiple layers of material and one or more scales for active component incorporation and dosage.

### BACKGROUND OF THE INVENTION

### 1. Field of Invention

The present invention relates to the field of measurement systems, more particularly, standardization systems for components having active ingredients or medicinal attributes.

### 2. Description of Related Art

Compounds or mixtures are used for a variety of purposes by millions of people on a daily basis. For example, cooking involves the mixture of various parts of a recipe. Whether the end result is a heterogeneous or homogenous mixture, the different components that go to make the end result all have to be evaluated and measured for their desired expression.

Pharmaceuticals are another example of a vital field involving detailed measurements of different components during manufacture and administration. One must be very precise in the measurements and concentrations of the various substances as the resulting medicinal effects can be life changing.

Currently, the area of medicine and cooking overlap in the administration of medicinal Tetrahydrocannabinol (THC) and cannabinoids (CBDs). While a traditional approach to administering has been to inhale THC and CBDs through combustion via smoking, the THC and CBDs are still potent when incorporated into a recipe resulting in an edible. Patients benefit greatly from the positive effects of THC and CBDs, but often do not wish to, or are unable to smoke these substances. Some patients, particularly minors, are incapable of smoking, yet require administration of THC and CBDs.

Often, a user or patient wanting to establish a specific amount of the active ingredient for their consumption is left to guess or perform technical and complicated mathematical computations for the appropriate amount of the component. This situation is assuming that an appropriate value has been given to the component. It is not uncommon for a user or patient to be left to trial and error processes when the effect of the active or medicinal compounds are present in unknown amounts.

Tinctures, oils, lotions, and edibles are commercially available where by a manufacturer, with large amounts of capital can have these products developed and tested in a laboratory. This process is done to the exclusion of an individual's autonomy. Not everyone has the technical ability nor the financial resources to properly identify how to, or how much, medicine to include in a particular combination, mixture, or recipe.

Based on the foregoing, there is a need in the art for a system that will allow a user to identify the specific amounts of THC, CBDs, and other compounds in a substance such that they can be used in new or known recipes. Further, there is a need for a system that will ensure the end result of combined substances is known and that the composition of medicinal components within the end result can be easily calculated for appropriate dosing.

### SUMMARY OF THE INVENTION

In an embodiment, a method for recipe standardization has the steps of a user creating a profile having one or more user characteristics, wherein the one or more user characteristics comprises user tolerance. A system prompting user input, wherein the system provides one or more options for recipe creation. The user selecting at least one of the one or more options for recipe creation; the user selecting desired effect of the recipe, wherein the desired effect is selected from one or more scales. The system determining the composition of the recipe. An algorithm creating a threshold, wherein the algorithm accounts for the user characteristics and the desired effect. The algorithm adjusting the amount of one or more of recipe components, wherein the adjusting is based on a deviation of the user characteristics from the threshold. The system generating instruction for recipe preparation.

In an embodiment, one or more of the recipe components is a medicinal compound having one or more active components. For example, the medicinal compound may be Tetrahydrocannabinol (THC), or the medicinal compound is a cannabinoid (CBD). In an alternative embodiment, the medicinal compound has inactive ingredients.

In an embodiment, the recipe standardization system involves the step of the user inputting the percent by weight of the active components of the medicinal compound into the system and the algorithm accounting for the percent by weight of the active components in the compound.

In an embodiment, one or more user characteristics ay include: gender, height, weight, age, physical condition, medical history, and family medical history.

In an alternative embodiment, a device analyzes a sample of one or more recipe components. For example, the a sample of the recipe component is dissolved within the device, wherein the solution comprises the one or more active components, and wherein the analysis identifies the percent of the one or more active components by weight. In an embodiment, the device communicates the analysis to the system where the algorithm adjusting the amount of the one or more recipe components, wherein the amount of the medicinal compound is adjusted based on the percent by weight of the one or more active components.

In an alternative embodiment, the system communicates one or more instructions to an automated recipe preparation system such that the automated recipe preparation system mixes raw recipe components and the automated recipe preparation system prepares the mixture of the recipe components, wherein the raw recipe components are converted to homogenous or heterogeneous edible results during the preparation.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a flowchart of a method for dosage standardization, according to an embodiment of the present invention.

FIG. 2 is a system for dosage standardization, according to an embodiment of the present invention.

FIG. 3 is a system for dosage standardization, according to an embodiment of the present invention.

### DETAILED DESCRIPTION OF PREFERRED EMBODIMENTS

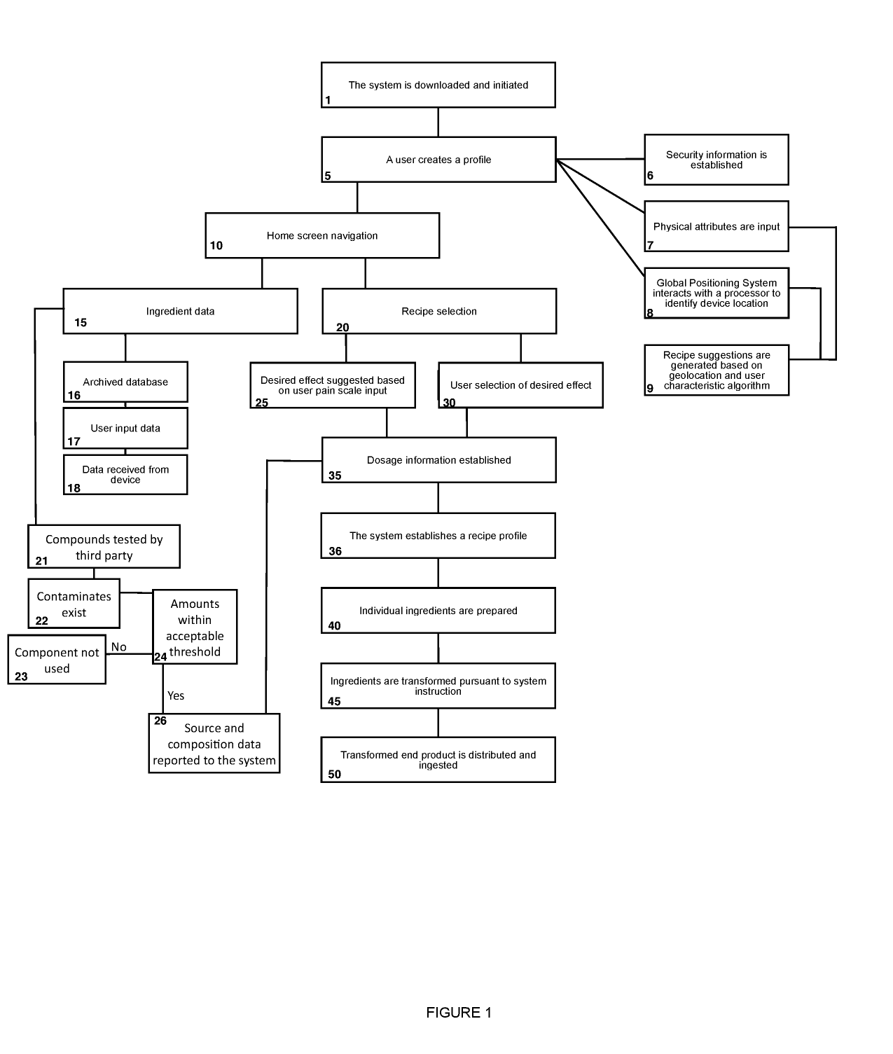

Preferred embodiments of the present invention and their advantages may be understood by referring to FIGS. 1-3, wherein like reference numerals refer to like elements.

In an embodiment, the system operates on a mobile device. In another embodiment, the system is network connected, on a website or web app for example, to enable convenient user interaction.

In describing the user interface to the system, and with reference to FIG. 1, once the system is initiated (step 1) for the first time, a user creates a profile (step 5) that includes personal information (step 7) and geographical information (step 8). The profile also includes options for setting privacy restraints (step 6) where a user may indicate an approval to release certain aspects of the profile for networking purposes or suggestions (step 9) to the user of specific recipes and substances. The profile section will also allow for a user to input an image or icon for recognition. The profile creation also involves establishing a username and password, which are used for subsequent login to the system.

In an embodiment, the system locates a specific user via a Global Positioning System (GPS) (step 8). A processor interacts with a GPS device to establish coordinates and input those coordinates into the system. When establishing a profile (step 5), a user has an options to turn the GPS component on or off during use of the system.

In an embodiment, the profile creation (step 5) includes user input of physical attributes 7 such as gender, height, weight, and age. In an alternative embodiment, the system offers multiple options for selection of the user body type. The profile further includes optional input for aspects of the user medical history. The user, in text fields or pre-established selection icons, indicates instances of chronic and acute disease such as physical ailments, mental disorders, cancer, cardio vascular diseases, and pulmonary diseases. Subsequent to profile creation, when the user logs into the system, the profile is associated with their account and the information contained therein is recalled. The system presents a platform with a plurality of options from which the user can choose, such as a home screen navigation (step 10).

In an embodiment, the platform is home screen navigation (step 10) where displayed information is generated based upon the user profile. For example, with the GPS initiated, the home screen may display selected regional dishes, inspiration, or suggestions as options for a user to select and proceed with making. In another embodiment, the home screen displays a plurality of different options for selecting a use of the desired ingredients. For example, options may include ingredient data (step 15) and recipe selection (step 20).

In an embodiment, an algorithm is used to determine the specific composition of a recipe using selected ingredients. The algorithm includes the user input information such as physical attributes and subjective user experience with the selected ingredients. For example, a user will initiate the app, after having established a profile. The user will then be directed to the home screen (step 10) having multiple options to select specific categories and subcategories of recipes (step 20). The categories and sub categories are divided based upon the genera of dish, such as sweet or savory. After the user has selected the category of recipe, the user inputs the specific percentages of active compounds of their ingredients, such as the THC and CBD content of a particular strain of cannabis.

The system establishes baseline concentration values based on user input information for the profile creation. These baseline values are then shared with the system algorithm to create a standard level for medicinal compound inclusion. When the user profile is adjusted, the algorithm compensates for deviations from the baseline levels to account for fluctuations, increases, or decreases in user characteristics. For example, after the algorithm has established baseline values, the characteristic for user weight is accounted for. If the user weight is above a threshold baseline level, concentrations of required medicinal compounds may be increased or adjusted according to the desired result. Another example is the characteristic directed to user experience or tolerance for the medicinal compound that can result in the algorithm adjusting ingredient values based on the changing tolerance.

In another embodiment, the system locates the specific user via GPS location of their mobile device. After a location has been established and the system has accounted for the user input information, suggestions are presented on nearby locations of various ingredients including locations of suppliers of the medicinal or active components of the recipe. For example, a recipe may require flour, yeast, butter, sugar, and cannabis oil. The system presents the user with an option to search nearby vendors. After selecting this option, the system will interact with accessible networks including wireless internet and cellular networks to search and cross reference locations having the necessary ingredients. Prices may be provided along with options to purchase, pay, and set a pickup time. Another example is when the system will direct the patient to local or nearby pharmacies or dispensaries (mobile or fixed)/purveyors/growers/labs/brokers/any and all legal participants in the industry of the recommended products (raw, refined and/or even packaged) and ready for legal distribution. In another embodiment, a supplier will have the opportunity to verify the user's use of the medicinal component and setup a delivery. The supplier will have authorized access to the user's location for setting the logistical details of the delivery.

In an embodiment, the user is provided a unique identifier. The unique identifier allows for rapid recall and input of user information based on any information associated with the unique identifier. For example, the user information associated with the unique identifier may include medical diagnosis, expert recommendations, previous user input, and system generated suggestions for scaling the inclusion of the medicinal compound.

In another embodiment, the unique identifier operates as a marker for information to be attributed to a user. In certain situations, the user will still have a sample of a medicinal component evaluated by various third party agencies, for example a laboratory. These third parties will evaluate the sample and compile specific data on the sample. The third parties are able to relay their data to the user through verification of their unique identifier where the third party is able to supply the data to the system remotely and the data is stored and accessible by the user's system on a mobile device. For example, a user may submit a sample of cannabis to a third party for study. When the third party has completed the study, the information obtained is entered into the system by the third party and linked to the unique identifier. The user is then able to review the information. If the sample is to be used in a recipe, the system will automatically adjust for the data input by the third party. In an embodiment, the data obtained and input into the system identifies the geographic region, any identifiable mutations, and history of the medicinal compound.

In another embodiment, the system on a mobile device communicates with one or more other systems to allow for user connectivity with experts operating under an expert or supervisory portal. The expert, for example a physician, is able to link to the user system and review the material provided by the user. The expert may provide real-time advice or suggestions for the user.

In an alternative embodiment, the specific THC and CBD content is unknown and a user may select from a cataloged of average THC and CBD content for archived (step 16 strains. The user, selecting an option from a subjective scale, establishes the determination of the desired result or effect (step 30) of THC and CBD in the recipe.

In an embodiment, an acceptable dosage value is unknown and the user is unaware of the appropriate amount of a component to consume for a desired effect. The system extracts data contained within a storage media device or stored one or more networks servers, where the data includes user input regarding physical and mental characteristics as well as information about the medicinal or active components to be incorporated into the recipe and ingested by the user. The extracted data is then incorporated into the system through one or more algorithms executed by one or more processors. Information is then output to the user regarding the suggested appropriate dosage value.

In an embodiment, the system gathers information regarding the origin, disposition, and displacement of the medicinal compound. This information is input by either the user, or a third party. A third party is able to engage the system to provide various aspects of the manufacture of the medicinal compound. The third party inputs this information into the system along with a standardized evaluation of a sample. This evaluation is used for cross referencing of information by users who may have acquired an amount of the third parties product. For example, a grower and/or processor of cannabis is the third party and inputs objective and subjective information about the cannabis they produce, which may include a sample evaluation. If a user has a known or unknown sample evaluated, the system will cross reference the user sample data with a database including the third party evaluation for further verification of the origin and processing of the user sample. In another embodiment, one or more growers is assigned a unique identification that is held within the system and stored in a database. The system recalls the unique identification for subsequent grower or third party interaction with the system.

In another embodiment, the system engages a photo or video recording component of the mobile device. The system is able to retrieve information based on a visual input from the photo or video recording component. In an embodiment, a user selects a medicinal compound having a unique packaging. The unique packaging comprises an identifier such as a barcode, QR code, image, text, serial number etc. The user inputs the identifier into the system and the system retrieves information based on the identifiers of the medicinal compound. The system then auto-populates the known data into the algorithm for consideration of the characteristics of the medicinal compound for inclusion into a recipe.

In an embodiment a scale is provided for a user to establish the desired effect (step 25) of the recipe. For example, the scale may include a range of numbers that correspond with varying degrees of medicinal effect. In another example, a user will select from icons, images, or text, all of which would correspond with a desired effect.

In an alternative embodiment, the archived information of THC and CBD data (step 16) is manipulated and uploaded by a user (step 17) into their specific system. This information can be made available, at the user's command, to other users through an established network. When the mobile device is connected to a network such as wireless internet, Bluetooth, or other interactive platforms, the data can be shared with other users (step 18).

In an embodiment, the user has selected a desired recipe with a desired effect (step 30) The system then utilizes an algorithm to account for the output quantity, effect, selected recipe, the percent of active components of the ingredients, and state of the compound being included for the establishment of dosage information (step 35). The desired effect, output quantity, and the percent of active ingredients, such as THC, are factors of the amount of ingredients included in the recipe profile (step 36). The state of the compound may be a solid, such as dry herbs, or liquid oil. A recipe is then displayed with step-by-step instruction for a user to follow. Individual ingredients of the recipe are prepared (step 40). Based on the recipe, the included ingredients are converted into the end result (step 45). The end result is then distributed or ingested by the user (step 50).

In an embodiment, the system includes a device for testing the content of active components of various ingredients. The testing device has a platform, a power source, a processor, one or more buttons, and a display screen. In an alternative embodiment, the device is handheld and will engage the system using wireless connectivity, such as Bluetooth or wireless Internet. The platform is in electrical communication with the processor. In use, a compound, such as a sample of cannabis, is placed on the platform. A solution may be used to dissolve the sample for evaluation. Through extraction, the solvent separates the THC from the cannabis and the platform is configured to identify the level of THC content of the sample using refractive processes or litmus type uptake of the dissolved THC. The processor accepts the information from the platform and displays the information on the screen. The device then sends the information to the system for consideration of the amount to add in the recipe.

In an embodiment, the system maintains information regarding compounds having potential negative impact on the user. Compounds having negative impact, such as contaminates, may include pesticides or other elements incorporated into the various components throughout the growing, manufacturing, and distribution stages. An example of a compound having a negative impact is Malathion and similar pesticides used during the growing process for many crops including cannabis. The information on compounds having a negative impact is stored within the system or on a database for subsequent recall when sample evaluation is submitted. For example, the user or a third party tests the sample (step 21) and the result input into the system. Threshold values for acceptable amounts of negative impact compounds are then recalled for consideration of exceeding the threshold limit of discovered negative compounds within the sample. If contaminates exist (step 22), the system will determine if the amount of contaminates within the sample are within acceptable threshold amounts (step 24). This system presents the amount of negative compound with a recommendation for maximum intake values to the user for consideration. If the amount of contaminate is above the threshold value, the component is not used (step 23) in the recipe creation. If the values of contaminate are acceptable the information derived from the third party is reported to the system (step 26).

In an embodiment, the device has a fluid reservoir containing one or more solvents. The fluid reservoir is in communication with a pump to force or extract the fluid from the reservoir to the sample. A platform houses the sample and a cap sealingly engages the platform creating tight seal to the platform preventing unwanted evaporation. The platform also acts as a scale to measure the weight of the sample at one or more times during the testing process. The platform is in electrical communication with one or more processors and one or more power supplies. The processor is configured to identify the amount of THC and CBD extracted through the solvent-sample mixture. The dissolved THC and CBD are either weighed by the platform to determine their percent by weight, or the solution containing THC and CBD is then analyzed through a spectrometer.

In an alternative embodiment, the system instructs manufacture and fabrication of a recipe through an automated system. Ingredients are placed in individual holding containers in communication with the automated system. When the user selects a recipe and establishes the desired effect of the active ingredients within the recipe, the system communicated with the automated manufacture system. The automated manufacture system selects ingredients from their individual containers and manipulates the ingredients according to the recipe. When ingredients are combined, they form heterogeneous or homogenous mixtures that are then subjected to specific temperatures for cooking or baking. When the process is complete, the final product is ready for consumption pursuant to the specifications of the established recipe from the user in the system.

In an embodiment, the system assigns a value for algorithm incorporation whereby the processor executes the algorithm with a plurality of factors include the user's input and attributes as well as the attributes and characteristics of the component being incorporated into the recipe. For example, the system will account for information such as the specific strain of the medicinal/active compound (i.e. strain of cannabis), user ailments and diseases, any identified symptoms or symptoms that the system unilaterally associates with the ailment, and the known attributes for indicia or sativa strains.

In another embodiment, a user inputs data into the system where the processor executes the algorithm accounting for various disease or ailment symptoms. The system communicates with one or more databases containing general information relating to the user input disease or ailment. The information is retrieved from the database by the system and incorporated into he algorithm. The user may be presented with the general information to be input by the system as an opportunity to allow or decline the incorporation of the database material. In this embodiment, the database supplies information the system identifies as relevant to establish a more precise output.

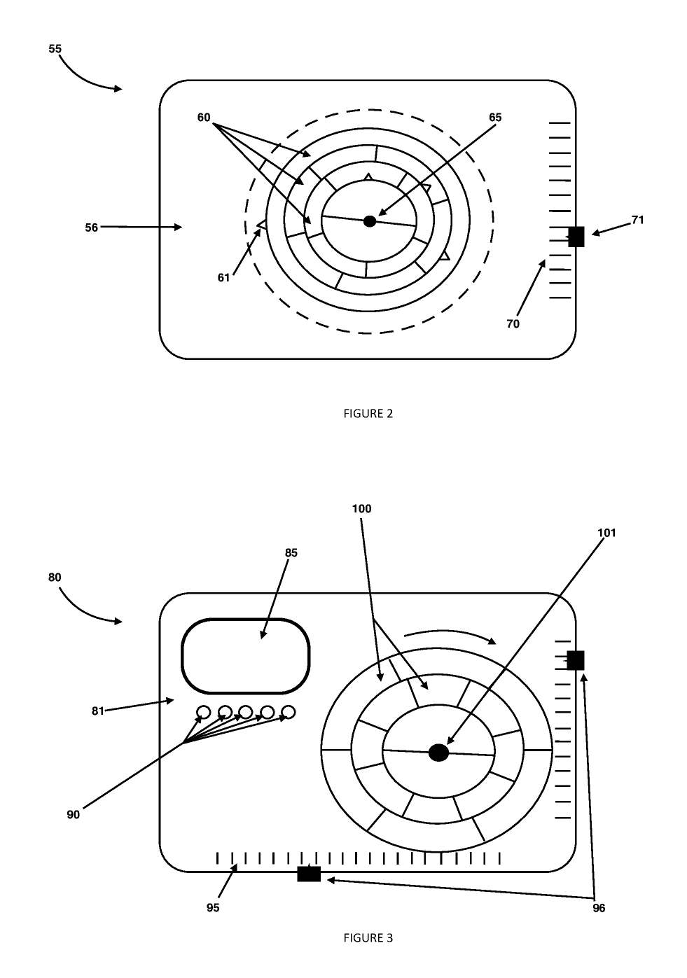

In an alternative embodiment, illustrated by FIG. 2, the system operates as an apparatus 55 having a surface plate 56 and one or more layers 60 of material in communication with the surface plate 56. Each layer of material is designated with a plurality of sectors and each layer has an indicator portion 61 allow for user manipulation of the layer to indicate a response.

In an embodiment, the system has a hub 65 to connect the surface plate 56 with each of the layers 60. The layers 60 of material are coaxially aligned concentric shapes. The size of the layers decreases as from the most proximal layer, relative to the surface plate 56, to the distal layer.

In an embodiment the apparatus has one or more scales 70 in communication with the surface plate 56. A selector tab 71 is slidably engaged with the one or more scales 70 such that the selector tab 71 is able to slide along the scale 70 to selected stopping points on the scale 70. In another embodiment, the scale is in communication with the apparatus such that when the selector tab is moved into position to identify a specific value on the scale 70, the scale 70 adjusts the overall parameter of the apparatus 55 for user interaction. The different points on the scale 70 correspond to values of measurement for the amount of an active or medicinal compound within a component or sample.

In an embodiment, the apparatus is used in multiple steps. First, a parameter is established by the user sliding the selector tabs to a set point corresponding to known values of the amount of an active or medicinal compound within a sample. For example, if a sample of cannabis has 27% THC by weight, the scale is set to identify 27% THC content. The apparatus will then be calibrated based on the specific amounts of the known active or medicinal compounds of component. Next, the user engages the outermost layer of the apparatus rotating the outermost layer to indicate the user response. In this step, the outermost layer comprises an initial inquiry directed to begin identifying relevant user characteristics or attributes. For example, the initial layer may be directed to the gender of the user and the user rotates the layer until the appropriate response is identified by an arrow pointing to the user response. Then the user continues through each layer in a similar manner of selecting a response to the inquiry presented by the respective layer. For example, subsequent layers may be directed inquiries such as age, weight, select known health issues, experience with the component (history of use of the medicinal compound), etc. Each layer is rotated individually in response to the respective inquiry presented. Finally, after the user has completed interactions with each layer, a resulting output value is identified and displayed to the user, whereby the output value corresponds to an amount of the compound to be consumed or included in the creation of a recipe.

In an alternative embodiment, illustrated by FIG. 3, the apparatus 80 has a power source (not shown), at least one processor (not shown), a plurality of buttons 90, and a screen 85 to display text or images. The surface plate 81 is in electrical communication with the one or more layers 100. For example, the hub 101 is a communication intermediary such that as each layer is rotated to a desired response the response is transmitted to the surface plate 81.

In another embodiment, the surface plate 81 has one or more algorithms to account for the selected response data. The processor executes the algorithm when information regarding user indicated responses to each of the inquiries presented by each of the layers 100 has been selected. Information from the sliding scales 95 relating to the known content of the medicinal or active compound is incorporated into the algorithm when one or more selector tabs 96, in electrical communication with the scales 95, are slid along the where the output data is amended or adjusted according to a parameter set by the identified amount of the medicinal or active component. In use, the user manipulates each layer of material 100 indicating the desired response to the inquiry presented and the response is electrically communicated to the processor where the algorithm accounts for the responses and any information submitted regarding the attributes of the sample. The algorithm then provides output data regarding the specific amount of the substance to be prepared for user consumption.

In another embodiment, the surface plate 81 is in communication with the system on a mobile device. Information input by the user through the one or more layers 100 of the surface plate 81 is communicated to the system on the mobile device where the data regarding amounts of the component, to be included in the recipe, are incorporated into the output data of the system on the mobile device.

In an alternative embodiment, the operation of the apparatus is digitized such that the function of the multiple layers 100, the surface plate, and the scales 95 are presented on a screen 85 of a mobile device and the user operates the apparatus through engaging the screen 85 with tactile contact.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A method for dosage standardization comprising the steps of:
a. a user creating a profile having one or more user characteristics, wherein at least one of the one or more user characteristics is user tolerance of an active ingredient;
b. a system prompting user input, wherein the system provides one or more options for recipe creation;
c. the user selecting at least one of the one or more options for recipe creation;
d. the user selecting desired effect of the recipe, wherein the desired effect is selected from one or more scales;
e. the system determining the composition of the recipe;
f. an algorithm creating a threshold, wherein the user characteristics and the desired effect are accounted for by the algorithm;
g. the algorithm adjusting the amount of one or more of recipe components, wherein the adjusting is based on a deviation of the user characteristics from the threshold;
h. the system generating instruction for recipe preparation.

2. The method of claim 1, wherein the one or more recipe components comprises a medicinal compound.

3. The method of claim 2, wherein the medicinal compound is Tetrahydrocannabinol.

4. The method of claim 2, wherein the medicinal compound is a cannabinoid.

5. The method of claim 1, further comprising the step of the user inputting the percent by weight of the active components of a medicinal compound into the system and the algorithm accounting for the percent by weight of the active components in the compound.

6. The method of claim 1, wherein the one or more user characteristics are selected from the group consisting of: gender, height, weight, age, physical condition, medical history, and family medical history.

7. The method of claim 2, wherein the medicinal compound comprises one or more active components.
a. a device analyzing a sample of the one or more recipe components, wherein the a sample of the recipe component is dissolved within the device, wherein the solution comprises the one or more active components, and wherein the analysis identifies the percent of the one or more active components by weight;
b. the device communicating the analysis to the system;
c. the algorithm adjusting the amount of the one or more recipe components, wherein the amount of the medicinal compound is adjusted based on the percent by weight of the one or more active components.

8. The method of claim 1, further comprising the steps of:
a. the system communicating the instruction to an automated recipe preparation system;
b. the automated recipe preparation system mixing raw recipe components; and
c. the automated recipe preparation system preparing the mixture of the recipe components, wherein the raw recipe components are converted during the preparing.

9. A system for standardizing a dosage comprising:
a. a base having a plurality of output values;
b. a plurality of layers, wherein the plurality of layers of are coaxially aligned, wherein the plurality of layers are concentric, wherein the plurality of layers rotate about a central axis relative to the base, and wherein each of the plurality of layers are rotatable to select at least one of the plurality of output values; and
c. at least one scale attached to the base having a plurality of indicator markings, wherein a first end of one or more tabs are slidingly engaged with the at least one scale, wherein a second end of the one or more tabs is connected to the plurality of output values, and wherein the one or more tabs adjust a baseline of the plurality of output values.

10. The system of claim 9, wherein the plurality of indicator markings correspond to an amount of at least one active compound within a recipe component.

11. The system of claim 10, wherein the at least one active compound is Tetrahydrocannabinol.

12. The system of claim 9, further comprising a hub extending from the plate through a center of each of the plurality of layers, wherein the hub is the central axis.

13. The system of claim 9, further comprising:
a. at least one power source;
b. a plurality of buttons; and
c. one or more processors,
wherein the power source is electrically connected to the one or more processors,
wherein the hub is in communication with the one or more processors, and
wherein a degree of rotation is communicated to the one or more processors.

14. The system of claim 13, wherein the system is in communication with a mobile device.

15. A system for standardizing a dosage comprising:
a. a power source;
b. one or more processors;
c. at least one visual display unit, wherein the at least one visual display unit, the one or more processors, and the power source are in electrical communication;
d. a base having a plurality of output values;
e. a plurality of layers, wherein the plurality of layers of are coaxially aligned, wherein the plurality of layers are concentric, wherein the plurality of layers rotate about a central axis relative to the base, wherein the central axis is in communication with the at least one processor, and wherein each of the plurality of layers are rotatable to select at least one of the plurality of output values; and
f. at least one scale attached to the base, the scale having a plurality of indicator markings,
wherein one or more tabs are slidingly engaged with the at least one scale,
wherein the at least one scale and the one or more tabs are in communication with the one or more processors, wherein the one or more tabs adjust a baseline of the plurality of output values, and wherein at least one of the plurality of output values is adjusted based on an angle of rotation of the plurality of layers.

16. The system of claim 15, wherein the plurality of indicator markings correspond to an amount of at least one active compound within a recipe component.

17. The system of claim 16, wherein the at least one active compound is Tetrahydrocannabinol.
